(19) United States
(12) Patent Application Publication
(10) Pub. No.: US 2026/0189884 A1
(43) Pub. Date: Jul. 2, 2026

(54) PATIENT SUPPORT APPARATUS HAVING WIRELESS SIDE CHANNEL FOR SERVICE TOOL

(71) Applicant: Hill-Rom Services, Inc., Batesville, IN (US)

(72) Inventors: Bryan M. Weidman, Columbus, IN (US); Eric D. Benz, Sunman, IN (US)

(73) Assignee: Hill-Rom Services, Inc., Batesville, IN (US)

(21) Appl. No.: 19/424,211

(22) Filed: Dec. 18, 2025

Related U.S. Application Data
(60) Provisional application No. 63/739,857, filed on Dec. 30, 2024.

(51) Int. Cl. H04W 4/33; A61G 7/05; G16H 40/67; H04W 76/14; H04W 84/12

(52) U.S. Cl. CPC H04W 4/33; A61G 7/05; A61G 7/0527; G16H 40/67; H04W 76/14; A61G 2203/12; H04W 84/12

(57) ABSTRACT

A patient support apparatus has a wireless side channel that is used for linking with a service tool, such as a tablet computer or laptop computer, to receive service updates for the patient support apparatus. The wireless side channel communicates wirelessly with the service tool while normal wireless communications between the patient support apparatus and a network of a healthcare facility are maintained. Thus, the patient support apparatus is able to continue to support a patient thereon while service updates are being installed. In one implementation, the wireless side channel uses WiFi Direct communications and while the network communications are WiFi communications.

The present application claims the benefit, under 35 U.S.C. § 119(e), of U.S. Provisional Ser. No. 63/739,857 , filed Dec. 30, 2024, which is hereby incorporated by reference herein in its entirety.

### BACKGROUND

The present disclosure relates to patient support apparatuses having circuitry that is accessible for service updates by a service tool such as a laptop computer or tablet computer. More particularly, the present disclosure relates to a system in which patient support apparatuses, such as patient beds, receive service updates via wireless communications with a service tool.

Currently, the most common way that patient support apparatuses, such as patient beds, receive service updates is via a physical connection with a service device, such as a tablet computer having a custom bed service application, via a cable like a Universal Serial Bus (USB) cable or a similar type of cable. In some cases, a cover of the patient bed needs to be removed to expose a port to which the service cable connects. Such activities around the patient bed tends to disturb the patient supported on the bed and can even necessitate that the patient be removed from the bed in some circumstances. It is also possible that the patient bed needs to be disconnected altogether from a network of the healthcare facility during service updates.

Some patient beds may receive service updates wirelessly but this requires use of the same wireless network that is used for wireless communications between the bed and the healthcare facility network. Accordingly, implementation of this type of wireless service updating also will typically require that the patient be removed from the bed during the service update process. What is needed, therefore, is a system in which service updates can be provided to a patient bed wirelessly without the need to disturb the patient and without the need to remove the patient from the bed. That is, it would be desirable for the service updates to be communicated wirelessly to the patient bed while the bed continues to operate normally to support the patient thereon and while the bed continues to communicate wirelessly with a network of the healthcare facility. Service technicians would also welcome a system in which service updates can be installed on patient beds without the need to enter the rooms in which the beds are located.

### SUMMARY

An apparatus, system, or method may comprise one or more of the features recited in the appended claims and/or the following features which, alone or in any combination, may comprise patentable subject matter:

According to a first aspect of the present disclosure, a system for use in a healthcare facility that may have a network that may include a plurality of wireless access points may be provided. The system may include a patient support apparatus that may have circuitry that may include a microcontroller that may be configured to communicate wirelessly via a first communication link with at least one wireless access point of the plurality of wireless access points. The microcontroller may be configured to execute operating instructions to control one or more functions of the patient support apparatus. The circuitry of the patient support apparatus may include at least one sensor that may be communicatively coupled to the microcontroller and that may be configured to detect at least one patient parameter that may be classified as protected health information (PHI). The system of the first aspect also may include a service tool that may be configured to communicate wirelessly with the microcontroller via a second communication link to provide service updates to the circuitry of the patient support apparatus. The circuitry may be configured to maintain the first communication link during communications with the service tool via the second communication link thereby permitting a patient to remain supported on the patient support apparatus while the service updates are provided to the circuitry of the patient support apparatus. The microcontroller may be configured so that the PHI may be wirelessly transmittable from the patient support apparatus via the first communication link but not via the second communication link.

In some embodiments of the first aspect, the first communication link may include a WiFi communication link and the second communication link may include a WiFi Direct communication link. If desired, the microcontroller may be included in a system on a module (SoM) that may have an antenna that may be used for both WiFi and WiFi Direct communications according to a time slicing protocol. Alternatively or additionally, the circuitry of the patient support apparatus of the first aspect may include a patient presence detector to determine whether the patient may be present on the patient support apparatus or absent from the patient support apparatus. If the patient is absent from the patient support apparatus while the service tool is in communication with the microcontroller via the WiFi Direct communication link, the time slicing protocol may be turned off automatically by the microcontroller such that the antenna may be dedicated for WiFi Direct communications with the service tool. Optionally, the service tool of the first aspect may be configured to permit a service technician to turn the WiFi communications of the WiFi communication link on and off based on commands that may be sent from the service tool via the WiFi Direct communication link.

The present disclosure further contemplates that the microcontroller of the first aspect may serve as a main microcontroller and the circuitry of the patient support apparatus may include a plurality of subordinate microcontrollers. In such embodiments, the service updates may be communicated to the main microcontroller from the service tool during a service communication session and then, after completion of the service communication session, the main microcontroller may be configured to promulgate the service updates out to corresponding ones of the subordinate microcontrollers to which the service updates may pertain.

In some embodiments of the system of the first aspect, a bed data communication module may be spaced from the patient support apparatus and may be mounted at a fixed location in a patient room in which the patient support apparatus may be located. If desired, the bed data communication module may be in wireless communication with one of the subordinate microcontrollers of the circuitry of the patient support apparatus via a third communication link and may be in communication with a nurse call system of the healthcare facility. Also if desired, the service tool may be configured to communicate service updates for the bed data communication module to the main microcontroller of the patient support apparatus during the service communication session and then, after completion of the communication session, the service updates for the bed data communication module may be sent to the bed data communication module via the one of the subordinate microcontrollers and the third communication link. In such arrangements, the first communication link may include a WiFi communication link, the second communication link may include a WiFi Direct communication link, and the third communication link may include a Bluetooth communication link. Alternatively or additionally, the main microcontroller may be configured to promulgate the service updates out to corresponding ones of the subordinate microcontrollers via any one or more of the following protocols: SPI, I²C, CAN, USB, RS-232, and RS-485.

More broadly, therefore, the present disclosure contemplates that a bed data communication module may be spaced from the patient support apparatus and may be mounted at a fixed location in a patient room in which the patient support apparatus may be located. Such a bed data communication module may be in wireless communication with the circuitry of the patient support apparatus via a third communication link and may be in communication with a nurse call system of the healthcare facility. If desired, the service tool may be configured to communicate service updates for the bed data communication module to the microcontroller of the patient support apparatus during a service communication session and then, after completion of the service communication session, any service updates for the bed data communication module may be sent to the bed data communication module via the third communication link. For example, the first communication link may include a WiFi communication link, the second communication link may include a WiFi Direct communication link, and the third communication link may include a Bluetooth communication link. Optionally, the circuitry may include a first antenna that may be used for wireless communications of both the first and second communication links and a second antenna that may be used for wireless communications of the third communication link.

In some embodiments of the system of the first aspect, the patient support apparatus may include a user input that may be selected to signal the circuitry to enter into a service tool discovery mode to link with the service tool via the second communication link. Alternatively or additionally, the patient support apparatus may include a plurality of user inputs that, in response to being selected in a predetermined sequence, may cause the circuitry to enter into a service tool discovery mode to link with the service tool via the second communication link. Further alternatively or additionally, the patient support apparatus may include a plurality of user inputs that, in response to being selected simultaneously, may cause the circuitry to enter into a service tool discovery mode to link with the service tool via the second communication link.

According to a second aspect of the present disclosure, a system for use in a healthcare facility that may have a network that may include a plurality of wireless access points may be provided. The system of the second aspect may include a patient support apparatus that may have circuitry that may include a microcontroller which may be configured to communicate wirelessly via a first communication link with at least one wireless access point of the plurality of wireless access points. Furthermore, the microcontroller of the second aspect may be configured to execute operating instructions to control one or more functions of the patient support apparatus. The system of the second aspect also may have a service tool that may be configured to communicate wirelessly with the microcontroller via a second communication link to provide service updates to the circuitry of the patient support apparatus. The circuitry of the second aspect may be configured to maintain the first communication link during communications with the service tool via the second communication link thereby permitting a patient to remain supported on the patient support apparatus while the service updates may be provided to the circuitry of the patient support apparatus. Still further, the service tool of the second aspect may be configured to permit a service technician to turn wireless communications of the first communication link on and off based on commands that may be sent from the service tool via the second communication link.

In some embodiments of the second aspect, the first communication link may include a WiFi communication link and the second communication link may include a WiFi Direct communication link. If desired, the microcontroller of the second aspect may be included in a system on a module (SoM) that may have an antenna that may be used for both WiFi and WiFi Direct communications according to a time slicing protocol. Alternatively or additionally, the circuitry of the patient support apparatus may include a patient presence detector to determine whether the patient may be present on the patient support apparatus or absent from the patient support apparatus. If the patient is absent from the patient support apparatus while the service tool is in communication with the microcontroller of the second aspect via the WiFi Direct communication link, the time slicing protocol may be turned off automatically by the microcontroller such that the antenna may be dedicated for WiFi Direct communications with the service tool.

Optionally, the circuitry of the patient support apparatus of the second aspect may include at least one sensor that may be communicatively coupled to the microcontroller and that may be configured to detect at least one patient parameter that may be classified as protected health information (PHI). Further optionally, the microcontroller may be configured so that the PHI may be wirelessly transmittable from the patient support apparatus via the first communication link but not via the second communication link.

The present disclosure further contemplates that the microcontroller of the second aspect may serve as a main microcontroller and the circuitry of the patient support apparatus may include a plurality of subordinate microcontrollers. In such embodiments, the service updates may be communicated to the main microcontroller from the service tool during a service communication session and then, after completion of the service communication session, the main microcontroller may be configured to promulgate the service updates out to corresponding ones of the subordinate microcontrollers to which the service updates may pertain.

In some embodiments of the system of the second aspect, a bed data communication module may be spaced from the patient support apparatus and may be mounted at a fixed location in a patient room in which the patient support apparatus may be located. If desired, the bed data communication module may be in wireless communication with one of the subordinate microcontrollers of the circuitry of the patient support apparatus via a third communication link and may be in communication with a nurse call system of the healthcare facility. Also if desired, the service tool may be configured to communicate service updates for the bed data communication module to the main microcontroller of the patient support apparatus during the service communication session and then, after completion of the service communication session, the service updates for the bed data communication module may be sent to the bed data communication module via the one of the subordinate microcontrollers and the third communication link. In such arrangements of the second aspect, the first communication link may include a WiFi communication link, the second communication link may include a WiFi Direct communication link, and the third communication link may include a Bluetooth communication link. Alternatively or additionally, the main microcontroller may be configured to promulgate the service updates out to corresponding ones of the subordinate microcontrollers via any one or more of the following protocols: SPI, I²C, CAN, USB, RS-232, and RS-485.

More broadly, therefore, the present disclosure contemplates that a bed data communication module of the second aspect may be spaced from the patient support apparatus and may be mounted at a fixed location in a patient room in which the patient support apparatus may be located. The bed data communication module of the second aspect may be in wireless communication with the circuitry of the patient support apparatus via a third communication link and may be in communication with a nurse call system of the healthcare facility. If desired, the service tool of the second aspect may be configured to communicate service updates for the bed data communication module to the microcontroller of the patient support apparatus during a service communication session and then, after completion of the communication session, any service updates for the bed data communication module may be sent to the bed data communication module via the third communication link. For example, the first communication link may include a WiFi communication link, the second communication link may include a WiFi Direct communication link, and the third communication link may include a Bluetooth communication link. Optionally, the circuitry of the second aspect may include a first antenna that may be used for wireless communications of both the first and second communication links and a second antenna that may be used for wireless communications of the third communication link.

In some embodiments of the system of the second aspect, the patient support apparatus may include a user input that may be selected to signal the circuitry to enter into a service tool discovery mode to link with the service tool via the second communication link. Alternatively or additionally, the patient support apparatus of the second aspect may include a plurality of user inputs that, in response to being selected in a predetermined sequence, may cause the circuitry to enter into a service tool discovery mode to link with the service tool via the second communication link. Further alternatively or additionally, the patient support apparatus of the second aspect may include a plurality of user inputs that, in response to being selected simultaneously, may cause the circuitry to enter into a service tool discovery mode to link with the service tool via the second communication link.

According to a third aspect of the present disclosure, a system for use in a healthcare facility that may have a network that may include a plurality of wireless access points may be provided. The system of the third aspect may include a patient support apparatus that may have circuitry that may include a microcontroller which may be configured to communicate wirelessly via a first communication link with at least one wireless access point of the plurality of wireless access points. The microcontroller of the third aspect may also be configured to execute operating instructions to control one or more functions of the patient support apparatus. The system of the third aspect may further include a service tool that may be configured to communicate wirelessly with the microcontroller via a second communication link to provide service updates to the circuitry of the patient support apparatus. The circuitry of the third aspect may be configured to maintain the first communication link during communications with the service tool via the second communication link thereby permitting a patient to remain supported on the patient support apparatus while the service updates may be provided to the circuitry of the patient support apparatus. Furthermore, the circuitry of the patient support apparatus of the third aspect may include an antenna that may be used for wireless communications over the first and second communication links according to a time slicing protocol. Still further, the circuitry of the patient support apparatus of the third aspect may include a patient presence detector to determine whether the patient is present on the patient support apparatus or absent from the patient support apparatus. If the patient is absent from the patient support apparatus of the third aspect while the service tool is in communication with the microcontroller via the second communication link, the time slicing protocol may be turned off such that the antenna may be dedicated for wireless communications only with the service tool over the second communication link.

In some embodiments of the system of the third aspect, the service tool may be configured to permit a service technician to turn wireless communications of the first communication link on and off based on commands that may be sent from the service tool via the second communication link. Optionally, the first communication link of the third aspect may include a WiFi communication link and the second communication link of the first aspect may include a WiFi Direct communication link. Alternatively or additionally, the circuitry of the patient support apparatus of the third aspect may include at least one sensor that may be communicatively coupled to the microcontroller and that may be configured to detect at least one patient parameter that may be classified as protected health information (PHI). The microcontroller of the third aspect may be configured so that the PHI may be wirelessly transmittable from the patient support apparatus via the first communication link but not via the second communication link.

The present disclosure further contemplates that the microcontroller of the third aspect may serve as a main microcontroller and the circuitry of the patient support apparatus of the third aspect may include a plurality of subordinate microcontrollers. In such embodiments, the service updates may be communicated to the main microcontroller from the service tool during a service communication session and then, after completion of the service communication session, the main microcontroller may be configured to promulgate the service updates out to corresponding ones of the subordinate microcontrollers to which the service updates may pertain.

In some embodiments of the system of the third aspect, a bed data communication module may be spaced from the patient support apparatus and may be mounted at a fixed location in a patient room in which the patient support apparatus may be located. If desired, the bed data communication module may be in wireless communication with one of the subordinate microcontrollers of the circuitry of the patient support apparatus via a third communication link and may be in communication with a nurse call system of the healthcare facility. Also if desired, the service tool may be configured to communicate service updates for the bed data communication module to the main microcontroller of the patient support apparatus during the service communication session and then, after completion of the service communication session, the service updates for the bed data communication module may be sent to the bed data communication module via the one of the subordinate microcontrollers and the third communication link. In such arrangements, the first communication link may include a WiFi communication link, the second communication link may include a WiFi Direct communication link, and the third communication link may include a Bluetooth communication link. Alternatively or additionally, the main microcontroller may be configured to promulgate the service updates out to corresponding ones of the subordinate microcontrollers via any one or more of the following protocols: SPI, I²C, CAN, USB, RS-232, and RS-485.

More broadly, therefore, the system of the third aspect may include a bed data communication module that may be spaced from the patient support apparatus and that may be mounted at a fixed location in a patient room in which the patient support apparatus may be located. The bed data communication module of the third aspect may be in wireless communication with the circuitry of the patient support apparatus via a third communication link and may be in communication with a nurse call system of the healthcare facility. If desired, the service tool of the third aspect may be configured to communicate service updates for the bed data communication module to the microcontroller of the patient support apparatus during a service communication session and then, after completion of the service communication session, any service updates for the bed data communication module may be sent to the bed data communication module via the third communication link. For example, the first communication link of the third aspect may include a WiFi communication link, the second communication link of the third aspect may include a WiFi Direct communication link, and the third communication link of the third aspect may include a Bluetooth communication link. Optionally, the circuitry of the third aspect may include a first antenna that may be used for wireless communications of both the first and second communication links and a second antenna that may be used for wireless communications of the third communication link.

In some embodiments of the system of the third aspect, the patient support apparatus may include a user input that may be selected to signal the circuitry to enter into a service tool discovery mode to link with the service tool via the second communication link. Alternatively or additionally, the patient support apparatus of the third aspect may include a plurality of user inputs that, in response to being selected in a predetermined sequence, may cause the circuitry to enter into a service tool discovery mode to link with the service tool via the second communication link. Further alternatively or additionally, the patient support apparatus of the third aspect may include a plurality of user inputs that, in response to being selected simultaneously, may cause the circuitry to enter into a service tool discovery mode to link with the service tool via the second communication link. Optionally, the patient presence detector of the third aspect may include one or more load cells of a weigh scale system of the patient support apparatus.

According to a fourth aspect of the present disclosure, a system for use in a healthcare facility having a network that may include a plurality of wireless access points is provided. The system of the fourth aspect may include a patient support apparatus that may have circuitry that may include a microcontroller which may be configured to communicate wirelessly via a first communication link with at least one wireless access point of the plurality of wireless access points. The microcontroller of the fourth aspect may be configured to execute operating instructions to control one or more functions of the patient support apparatus. The system of the fourth aspect further may include a service tool that may be configured to communicate wirelessly with the microcontroller via a second communication link to provide service updates to the circuitry of the patient support apparatus. The circuitry of the fourth aspect may be configured to maintain the first communication link during communications with the service tool via the second communication link thereby permitting a patient to remain supported on the patient support apparatus while the service updates are provided to the circuitry of the patient support apparatus. Still further, the system of the fourth aspect may include a bed data communication module that may be spaced from the patient support apparatus and that may be mounted at a fixed location in a patient room in which the patient support apparatus may be located. The bed data communication module of the fourth aspect may be in wireless communication with the circuitry of the patient support apparatus via a third communication link and may be in communication with a nurse call system of the healthcare facility. Also, the service tool of the fourth aspect may be configured to communicate service updates for the bed data communication module to the microcontroller of the patient support apparatus during a service communication session and then, after completion of the communication session, any service updates for the bed data communication module may be sent to the bed data communication module via the third communication link.

In some embodiments of the fourth aspect, the service tool may be configured to permit a service technician to turn wireless communications of the first communication link on and off based on commands that may be sent from the service tool via the second communication link. Optionally, the first communication link of the fourth aspect may include a WiFi communication link, the second communication link of the fourth aspect may include a WiFi Direct communication link, and the third communication link of the fourth aspect may include a Bluetooth communication link. Further optionally, the microcontroller may be included in a system on a module (SoM) having an antenna that may be used for both WiFi and WiFi Direct communications according to a time slicing protocol.

The present disclosure further contemplates that the circuitry of the patient support apparatus may include a patient presence detector that may determine whether the patient may be present on the patient support apparatus or absent from the patient support apparats. If the patient is absent from the patient support apparatus while the service tool is in communication with the microcontroller via the WiFi Direct communication link, the time slicing protocol may be turned off automatically by the microcontroller such that the antenna may be dedicated for WiFi Direct communications with the service tool. If desired, the patient presence detector of the fourth aspect may include one or more load cells of a weigh scale system of the patient support apparatus.

In some embodiments of the fourth aspect, the circuitry of the patient support apparatus may include at least one sensor that may be communicatively coupled to the microcontroller and that may be configured to detect at least one patient parameter that may be classified as protected health information (PHI). In such embodiments, the microcontroller may be configured so that the PHI may be wirelessly transmittable from the patient support apparatus via the first communication link but not via the second communication link. If desired, the PHI may also be transmittable from the patient support apparatus via the third communication link.

Optionally, the microcontroller of the fourth aspect may serve as a main microcontroller and the circuitry of the patient support apparatus of the fourth aspect may include a plurality of subordinate microcontrollers. Further optionally, the service updates may be communicated to the main microcontroller of the fourth aspect from the service tool during the service communication session and then, after completion of the service communication session, the main microcontroller may be configured to promulgate the service updates out to corresponding ones of the subordinate microcontrollers to which the service updates may pertain. Still further optionally, the bed data communication module may be in wireless communication with one of the subordinate microcontrollers of the circuitry of the patient support apparatus via the third communication link. After completion of the service communication session, the service updates for the bed data communication module of the fourth aspect may be sent to the bed data communication module via the one of the subordinate microcontrollers and the third communication link. If desired, the main microcontroller of the fourth aspect may be configured to promulgate the service updates out to corresponding ones of the subordinate microcontrollers via any one or more of the following protocols: SPI, I²C, CAN, USB, RS-232, and RS-485.

In some embodiments, the circuitry of the fourth aspect includes a first antenna that may be used for wireless communications of both the first and second communication links and a second antenna that may be used for wireless communications of the third communication link. Optionally, the patient support apparatus of the fourth aspect may include a user input that may be selected to signal the circuitry to enter into a service tool discovery mode to link with the service tool via the second communication link. Alternatively or additionally, the patient support apparatus of the fourth aspect may include a plurality of user inputs that, in response to being selected in a predetermined sequence, may cause the circuitry to enter into a service tool discovery mode to link with the service tool via the second communication link. Further alternatively or additionally, the patient support apparatus of the fourth aspect may include a plurality of user inputs that, in response to being selected simultaneously, may cause the circuitry to enter into a service tool discovery mode to link with the service tool via the second communication link.

According to a fifth aspect of the present disclosure, a method of providing service updates to a plurality of patient beds may be provided. Each patient bed may be situated in a respective patient room of a plurality of patient rooms of a healthcare facility, for example. The method of the fifth aspect may include transporting a service tool to a first position within a hallway situated between the patient rooms. The service tool of the fifth aspect may be within a communication range of a first subset of the plurality of patient beds. The method of the fifth aspect further may include operating the service tool to establish wireless communication links with a designated number of the first subset of the plurality of patient beds. The designated number may be less than a total number of the patient beds in the first subset. The method of the fifth aspect also may include transmitting the service updates wirelessly to the designated number of the first subset of the plurality of patient beds over the wireless communication links while each of the designated number of the first subset of the plurality of patient beds may continue to support a corresponding patient thereon and while the designated number of patient beds each may maintain a respective second wireless communication link with a network of the healthcare facility. After the service updates have been transmitted to the designated number of the first subset of the plurality of patient beds of the fifth aspect, the method further may include transporting the service tool to a second position within the hallway so that the service updates can be provided to another designated number of patient beds from a second subset of the plurality of patient beds.

In some embodiments of the method of the fifth aspect, operating the service tool to establish the wireless communication links with the designated number of the first subset of the plurality of patient beds may include determining signals strengths of wireless communications between the service tools and each patient bed of the first subset of the patient beds and may also include establishing the wireless communication links with the designated number of patient beds having the highest signal strengths. Alternatively or additionally, operating the service tool to establish the wireless communication links with the designated number of the first subset of the plurality of patient beds may include displaying on the service tool a menu that may identify the patient beds of the first subset and that may receive user inputs on the service tool indicating which of the patient beds of the first subset may be included in the designated number of the patient beds.

Optionally, displaying the menu on the service tool that may identify the patient beds of the first subset may include, for each of the patient beds of the first subset, displaying a software version number that may be currently installed on the respective patient bed. Further optionally, displaying the menu on the service tool that may identify the patient beds of the first subset may include, for each of the patient beds of the first subset, displaying information indicating whether a new version of bed operating software may be available for the corresponding patient bed. Still further optionally, displaying the menu on the service tool that may identify the patient beds of the first subset may include, for each of the patient beds of the first subset, displaying one or more of the following: bed model number, bed identification number, room number in which the patient bed may be located, and a signal strength icon that may indicate a signal strength of wireless communications between the service tool and the corresponding patient bed.

In some embodiments, the method of the fifth aspect further may include, for each of the patient beds within the first subset, detecting at least one patient parameter that may be classified as protected health information (PHI) with at least one sensor of the respective patient bed. In such embodiments, the PHI may be wirelessly transmittable from the respective patient bed via the corresponding second communication link but not via the first communication link. If desired, the first communication links of the fifth aspect each may include a WiFi Direct communication link and the second communication links of the fifth aspect each may include a WiFi communication link. Also if desired, each patient bed of the plurality of patient beds within the first subset of the fifth aspect may include a system on a module (SoM) having an antenna that may be used for both WiFi and WiFi Direct communications according to a time slicing protocol.

The present disclosure further contemplates that each patient bed of the plurality of patient beds of the fifth aspect may include a patient presence detector that may determine whether a respective patient is present on the corresponding patient bed or absent from the corresponding patient bed. If the patient is absent from the corresponding patient bed while the service tool is in communication with the corresponding patient bed, the method of the fifth aspect further may include turning off the time slicing protocol such that the antenna may be dedicated for WiFi Direct communications with the service tool. Alternatively or additionally, the service tool of the fifth aspect may be configured to permit a service technician transporting the service tool to turn the WiFi communications of each of the WiFi communication links on and off based on commands that may be sent from the service tool to the corresponding patient bed via the respective WiFi Direct communication link.

In some embodiments, the method of the fifth aspect further may include, for each of the patient beds of the first subset, receiving the service updates at a main microcontroller during a service communication session with the service tool. In such embodiments, each of the patient beds of the first subset may include one or more subordinate microcontrollers that may be in communication with the main microcontroller, and, after completion of the service communication session, the method may also include promulgating the service updates out to corresponding ones of subordinate microcontrollers of the corresponding patient bed to which the service updates may pertain.

If desired, the method of the fifth aspect further may include providing bed data communication modules in each of the patient rooms. Each bed data communication module of the fifth aspect may be spaced from the respective patient bed and may be mounted at a fixed location in the corresponding patient room. Each bed data communication module of the fifth aspect also may be in wireless communication with one of the subordinate microcontrollers of the corresponding patient bed via a third communication link and may be in communication with a nurse call system of the healthcare facility. Still further, the method of the fifth aspect may include communicating service updates for the respective bed data communication module to the main microcontroller of the corresponding patient bed during the service communication session and then, after completion of the communication session, the service updates for the bed data communication module may be sent to the bed data communication module via the one of the subordinate microcontrollers and the third communication link. In such embodiments, the first communication links of the fifth aspect each may include a WiFi Direct communication link, the second communication links of the fifth aspect each may include a WiFi communication link, and the third communication links of the fifth aspect each may incldue a Bluetooth communication link. Moreover, the main microcontroller of the fifth aspect may be configured to promulgate the service updates out to corresponding ones of the subordinate microcontrollers via any one or more of the following protocols: SPI, I²C, CAN, USB, RS-232, and RS-485.

More broadly, therefore, the present disclosure contemplates that the method of the fifth aspect further may include providing bed data communication modules in each of the patient rooms. Each bed data communication module may be spaced from the respective patient bed and may be mounted at a fixed location in the corresponding patient room. Furthermore, each bed data communication module may be in wireless communication with the respective patient bed of the fifth aspect via a third communication link and may be being in communication with a nurse call system of the healthcare facility. In such embodiments, the method of the fifth aspect further may include communicating service updates for the respective bed data communication module to the corresponding patient bed during a service communication session and then, after completion of the service communication session, communicating any service updates for the bed data communication module to the respective bed data communication module via the third communication link. Also in such embodiments of the fifth aspect, the first communication links each may include a WiFi Direct communication link, the second communication links each may include a WiFi communication link, and the third communication links each may include a Bluetooth communication link. Optionally, each patient bed of the fifth aspect may include a first antenna that may be used for wireless communications of both the first and second communication links and a second antenna that may be used for wireless communications of the third communication link.

In some embodiments, the method of the fifth aspect further may include establishing a wireless communication link between the service tool and a specific patient bed of the plurality of patient beds in response to selection of a user input on the specific patient bed which may cause the specific patient bed to enter into a service tool discovery mode to link with the service tool. Alternatively or additionally, the method of the fifth aspect further may include establishing a wireless communication link between the service tool and a specific patient bed of the plurality of patient beds in response to a plurality of user inputs of the specific patient bed being selected in a predetermined sequence which may cause the specific patient bed to enter into a service tool discovery mode to link with the service tool. Further alternatively or additionally, the method of the fifth aspect further may include establishing a wireless communication link between the service tool and a specific patient bed of the plurality of patient beds in response to a plurality of user inputs of the specific patient bed being selected simultaneously which may cause the specific patient bed to enter into a service tool discovery mode to link with the service tool.

Additional features, which alone or in combination with any other feature(s), such as those listed above, may comprise patentable subject matter and will become apparent to those skilled in the art upon consideration of the following detailed description of various embodiments exemplifying the best mode of carrying out the embodiments as presently perceived.

### BRIEF DESCRIPTION OF THE DRAWINGS

The detailed description particularly refers to the accompanying figures, in which:

FIG. 1 is a block diagram of a system for providing service updates from a service tool to a patient support apparatus, such as a patient bed, over a wireless side channel while the patient bed remains in wireless communication with a network of a healthcare facility;

FIG. 2 is a top plan view of a portion of a floorplan of a healthcare facility showing a service tool at a first position (in solid) in a hallway and in wireless communication range of four patient beds, and showing the service tool movable to second and third positions (in phantom) in the hallway for wireless communication with other patient beds;

FIG. 3 is an example of a first screen shot that appears on a display of the service tool during use, the first screen shot including a table of information regarding the patient beds which are within the communication range of the service tool;

FIG. 4 is an example of a second screen shot that appears on the service tool after a SELECT BEDS TO UPDATE button of the first screen shot has been selected by a user to indicate that service updates are desired for at least one of the patient beds;

FIG. 5 is an example of a third screen shot that appears on the service tool after the specific beds to receive service updates have been made on the second screen shot and after a SUBMIT button has been selected on the second screen shot by the user; and

FIG. 6 is an example of a fourth screen shot that appears on the service tool after the service update process has been completed for the selected patient beds.

### DETAILED DESCRIPTION

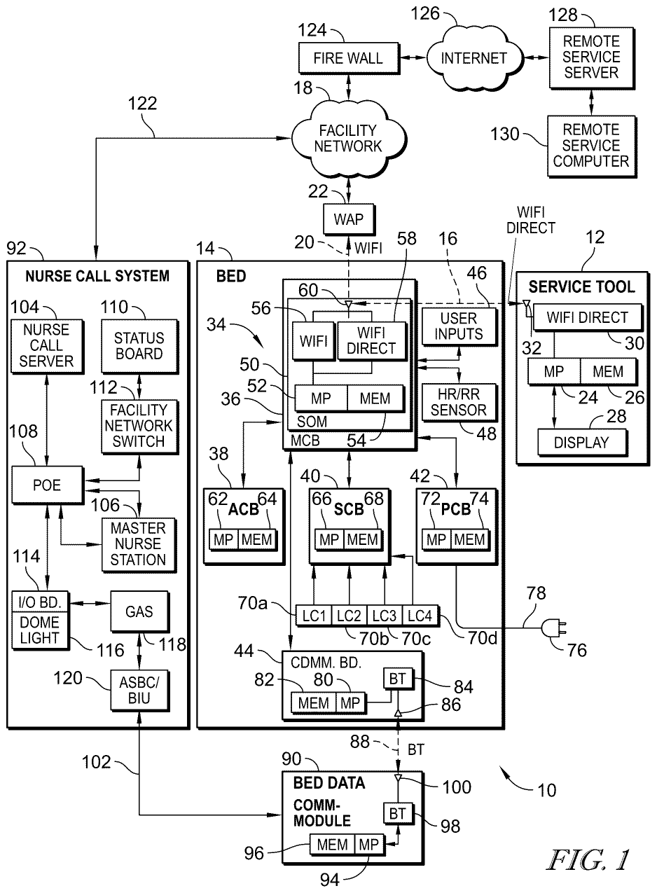

A system 10 for providing service updates from a service tool 12 to a patient support apparatus, such as a patient bed 14, over a wireless side channel 16 while the patient bed 14 remains in wireless communication with a network 18 of a healthcare facility over a wireless primary channel 20 is shown in FIG. 1. The service tool 12 is a portable device, such as a tablet computer or laptop computer or mobile phone, just to give a few examples, that is transported by a service technician throughout the healthcare facility to provide service updates wirelessly to respective patient support apparatuses 14. The services updates include, for example, updated software code to replace existing software code of the respective patient support apparatus 14, configuration data such as operating setpoints and/or device parameters, security patches to enhance or change security protocols for the patient support apparatus, and data lists such as lists of caregivers of the healthcare facility authorized to access certain features or functions of the patient support apparatus 14 or lists of rooms in the healthcare facility to account for new units or wings of the healthcare facility being added or removed during construction or renovation of the facility.

While the patient support apparatus illustrated herein is patient bed 14, it should be appreciated that the principles of the present disclosure may be implemented in connection with other types of patient support apparatus such as stretchers, chairs, wheel chairs, operating room tables, examination tables, imaging tables, and patient lifts including ceiling-support lifts and floor-supported lifts as long as such patient support apparatuses have circuitry with wireless communication capability.

In the illustrative example, the wireless side channel 16 between bed 14 and service tool 12 provides a first communication link that operates according to WiFi Direct communications technology, whereas the wireless primary channel 20 between bed 14 and network 18 provides a second communication link that operates according to WiFi communications technology. By using WiFi Direct technology as the wireless side channel 16 to connect service tool 12 to bed 14 for service updates, the wireless primary channel 20 using WiFi technology, which allows communication between bed 14 and a wireless access point 22 of network 18, is able to co-exist with the Wi-Fi Direct communication of channel 16 at the same time. This allows service technicians to establish the wireless first communication link over side channel 16 to bed 14 without needing to disturb the patient on bed 14 and without needing to remove any protective cover of the bed 14 for physical connection of the service tool 14 to circuitry of bed 14 using a hard cable, such as a Universal Serial Bus (USB) cable, for example. Thus, patient support apparatus 14 does not need to disconnect communications over the second wireless communication link of primary channel 20 with network 18 while service updates are provided over side channel 16 between the service tool 12 and bed 14.

Service tool 12 has control circuitry such as a microcontroller, which includes a microprocessor 24 and memory 26, as shown diagrammatically in FIG. 1. Service tool also 12 also includes a display 28 and a wireless communication transceiver 30 which, in the illustrative embodiment, comprises a WiFi Direct communication transceiver. Display 28 and transceiver 30 are in electrical communication with microprocessor 24. Display 28 comprises a touchscreen or graphical user interface (GUI), for example. Thus, microprocessor 24 receives user inputs from display 28 and provides visual outputs on display 28. In some embodiments, service tool 12 includes a keyboard (not shown) that is used by the service technician to provide inputs to microprocessor 24 in lieu of, or in addition to, the inputs provided through display 28.

In some embodiments, microprocessor 24, memory 26, and transceiver 30 of service tool 12 are provided on a single integrated circuit chip, such as a microcontroller chip or system on module (SoM) chip, although this is not required. Service tool 12 further includes an antenna 32 for transmission and reception of wireless signals over the first communication link 16. Antenna 32 is included in, or coupled to, transceiver 30, for example. In some embodiments, service tool 12 is also configured to communicate wirelessly with the one or more WAP's 22 of network 18 via WiFi and/or with transceivers of mobile phone towers (aka cellular towers) (not shown) outside of the healthcare facility using 4G, 5G, or similar such technologies. Such additional communication links of service tool 12 are omitted from FIG. 1.

Bed 14 includes an electrical system 34 that includes a wide variety of electrical components dispersed throughout the mechanical structure of bed 14. Thus, only some portions of the electrical system 34 of bed 14 are shown diagrammatically in FIG. 1. For example, the illustrative electrical system 34 of bed 14 includes a main control board (MCB) 36, an air control board (ACB) 38, a scale control board (SCB) 40, a power control board (PCB) 42, and a communications board 44. In the illustrative example, electrical system 34 of bed also includes user inputs 46 and one or more physiological sensors 48. User inputs 46 include, for example, patient control buttons accessible to a patient on bed 14 to operate a limited set of bed functions, caregiver control buttons that are accessible to caregivers (and generally inaccessible to the patient) to operate corresponding functions of the patient support apparatus, and a graphical user interface that is accessible to the caregiver to operate and control an expanded set of bed functions. In the illustrative example, sensor 48 comprises a heart rate (HR) and/or respiration rate (RR) sensor 48 to sense the HR and/or RR of the patient on bed 14.

The double headed arrows between MCB 36 and each of ACB 38, SCB 40, PCB 42, communications board 44, user inputs 46, and HR/RR sensor 48 in FIG. 1 are intended to represent the fact that these components of electrical system 34 form an on-board network of bed 14 with bidirectional communication links. It should be understood that electrical architectures in which ACB 38, SCB 40, PCB 42, communications board 44, user inputs 46, and HR/RR sensor 48 communicate with each other over other electrical pathways that do not include MCB 36 are within the scope of the present disclosure. For example, in some embodiments, bed 14 includes a controller area network (CAN) having a CAN bus to which each of MCB 36, ACB 38, SCB 40, PCB 42, communications board 44, user inputs 46, and HR/RR sensor 48 are coupled such that messages from each of MCB 36, ACB 38, SCB 40, PCB 42, communications board 44, user inputs 46, and HR/RR sensor 48 can be addressed to a selected recipient from among the others of MCB 36, ACB 38, SCB 40, PCB 42, communications board 44, user inputs 46, and HR/RR sensor 48. Furthermore, unidirectional communication links are provided between some components of electrical system 34 and others, in some embodiments. For example, in some embodiments, sensor 48 sends signals or messages to MCB 36 but does not receive any messages therefrom.

Still referring to FIG. 1, MCB 36 includes a microcontroller 50, illustratively a system on module (SoM) chip 50, having a microprocessor 52, memory 54, a first transceiver 56, and a second transceiver 58. In the illustrative example, transceiver 56 comprises a WiFi communication module and transceiver 58 comprises a WiFi Direct communication module 58. Microcontroller 50 further includes an antenna 60 that is shared by transceivers 56, 58 for wireless communications. Thus, antenna 60 is used by transceiver 58 to send and receive WiFi Direct messages over wireless side channel 16 to and from antenna 32 of transceiver 30 of service tool 12, respectively. Similarly, antenna 60 is also used by transceiver 56 to send and receive WiFi messages over wireless primary channel 20 to and from WAP 22 of network 18, respectively. A time slicing protocol (e.g., time division multiplexing) is implemented by microcontroller 50 to control when wireless messages are sent and received by antenna 60 over side channel 16 and when wireless messages are sent and received by antenna 60 over primary channel 20. The time slices back and forth between channels 16, 20 occur so rapidly that, for all intents and purposes, the communications over channels 16, 20 are considered to be occurring concurrently or at the same time.

ACB 38 has a microcontroller including a microprocessor 62 and a memory 64 which, in some embodiments, are included together on a single integrated circuit chip but this need not be the case. Microprocessor 62 of ACB 38 is used for control of pneumatic components of bed 14 such as blowers, compressors, and valves (not shown) that are used to inflate and deflate bladders of a mattress of bed 14. In some embodiments, ACB 38 has pressure sensors (not shown) mounted thereto to sense the air pressure within a particular bladder or zone of bladders of the mattress. For ease of illustration, the various pneumatic components associated with ACB 38 and the mattress of bed 14 are omitted from FIG. 1 but these are well known in the patient bed art.

SCB 40 has a microcontroller including a microprocessor 66 and a memory 68 which, in some embodiments, are included together on a single integrated circuit chip but this need not be the case. Microprocessor 66 of SCB 40 processes signals that are received by SCB 40 from four load cells 70a, 70b, 70c, 70d to determine the patient's weight and/or position on bed 14. For example, in some embodiments, each of load cells 70a, 70b, 70c, 70d has a strain gage included in a Wheatstone bridge resistor arrangement on a load cell block that deflects under the particular load imparted on the respective load cell 70a, 70b, 70c, 70d as is known in the art. The load cells are mounted to a weigh frame of the bed and support an intermediate frame that, in turn, supports articulatable deck sections that support the mattress of bed 14.

The signals produced by the load cells 70a, 70b, 70c, 70d are analog signals (e.g., voltage and/or current) which are fed to one or more analog-to-digital converters (ADC's) (not shown) of SCB 40. Digital outputs from the one or more ADC's are fed to microprocessor 66 which, in turn, determines the overall weight of the patient supported on the mattress of bed 14. The microprocessor 66 is also able to determine the patient's position, such as by determining a center of gravity of the patient based on the individual load cell readings as is known in the art. For ease of illustration, some of the various scale control circuit components associated with SCB 40 of bed 14 are omitted from FIG. 1 but these are well known in the patient bed art. In other embodiments, microprocessor 54 of microcontroller 50 determines the overall patient weight and/or position of the patient on bed 14 based on messages from SCB regarding the individual load cell readings.

Similar to ACB 38 and SCB 40, PCB 42 has a microcontroller including a microprocessor 72 and a memory 74 which, in some embodiments, are included together on a single integrated circuit chip, but this need not be the case. Microprocessor 72 of PCB 38 is used for control of various power detection, voltage conversion, battery charging, and power control components of bed 14. Thus, PCB 42 includes, for example, one or more transformers, rectifiers, voltage converters, voltage dividers, voltage regulators, resistors, inductors, capacitors, current sensors, and the like, at the discretion of the bed designer. A power plug 76 at an end of a power cord 78 of bed 14 is configured to plug into a standard alternating current (AC) receptacle of the healthcare facility to provide standard AC power (e.g., 120 Volts AC (VAC), 60 Hertz (Hz) power in the US) to PCB 42 which, in turn, converts the standard AC power into direct current (DC) voltages at desired levels, such as 5 VDC for operating integrated circuity chips, 24 VDC for powering motors (e.g., linear actuators) and/or air sources of bed 14, and the like. For ease of illustration, the various power control components associated with PCB 42 of bed 14 are omitted from FIG. 1 but these are well known in the patient bed art.

With continued reference to FIG. 1, communications board 44 includes a microcontroller having a microprocessor 80 and a memory 82 which, in some embodiments, are included together on a single integrated circuit chip, but this need not be the case. Microprocessor 80 controls various communication functions of bed 14 such as controlling audio feeds from a television or from a remote component such as a phone handset located at a master nurse station, for example. Thus, in some embodiments, bed 14 includes one or more speakers through which a selected audio feed is annunciated. In general, if a caregiver is speaking to a patient, the audio feed from the television is muted or turned off. Bed 14 also includes a microphone in some embodiments to permit a patient to speak to a caregiver at a remote location. Communications board also controls signals and messages that are sent to a nurse call system 92 of the health care facility. These signals and messages include, for example, bed status information regarding the state of various features of bed 14 such as caster brake status (e.g., braked or unbraked), siderail statuses (e.g., raised or lowered), bed upper frame position (e.g., in lowest position or not in lowest position), angle at which a head section of a mattress support deck is inclined (aka head-of-bed (HOB) angle), bed exit/patient position monitoring (PPM) system status (e.g., armed or disarmed; alarming or not alarming); etc., as well as other information such as bed model number, bed serial number, mattress status data, error conditions, plugged/unplugged status of power plug 76, etc. Nurse call system 92 is a subsystem of the overall system 10 of the healthcare facility. In some embodiments, a nurse call cable (not shown), such as a 37-pin cable, provides a wired connection between communications board 44 and nurse call system 92.

Illustrative communications board 44 further includes a Bluetooth (BT) radio (aka BT transceiver) 84 with a BT antenna 86. In some embodiments, microprocessor 80, memory 82, BT transceiver 84, and antenna 86 are included on a single integrated circuit chip such as a SoM chip, but this need not be the case. Antenna 86 is used by transceiver 84 to send and receive BT messages over a secure BT channel 88 to a bed data communication module 90 after a pairing operation is performed between the circuitry of bed 14 and module 90. Bed data communications module 90 is mounted at a fixed location, such as on a room wall of the patient room in which bed 14 is located, or on a wall of an architectural unit that, in turn, is mounted on the room of the patient room. Such architectural units include head wall units or service chases or columns having medical gas outlets and electrical outlets, for example.

To facilitate the BT communications over BT channel 88, bed data communications module 90 includes a microcontroller having a microprocessor 94 and a memory 96 which, in some embodiments, are included together on a single integrated circuit chip, but this need not be the case. Bed data communications module 90 further includes a Bluetooth (BT) radio (aka BT transceiver) 98 with a BT antenna 100. In some embodiments, microprocessor 94, memory 96, BT transceiver 98, and antenna 100 are included on a single integrated circuit chip such as a SoM chip, but this need not be the case. Antenna 100 is used by transceiver 98 to send and receive BT messages over secure BT channel 88 to antenna 100 and BT radio 84 of communications board 44 of bed 14. Bed data communications module 90 is, in turn, connected to nurse call system 92 by a nurse call cable 102. Thus, bed data communications module 90 permits wireless communication between bed 14 and nurse call system 92 via the wireless communication link provided over BT channel 88 in lieu of the standard nurse call cable that is used for providing a wired communication link between bed 14 and nurse call system 92.

In some embodiments, bed 14 is the CENTRELLA® Smart+Bed available from Hill-Rom Company, Inc. of Batesville, Indiana, U.S.A. Additional details of such embodiments of bed 14 can be found, for example, in U.S. Pat. No. 10,517,784 which is hereby incorporated by reference herein in its entirety for all that it teaches to the extent not inconsistent with the present disclosure which shall control as to any inconsistencies. Furthermore, in some embodiments, bed data communication module 90 is the READYCONNECT™ wall unit which is also available from Hill-Rom Company, Inc. Additional details of such embodiments of module 90 can be found, for example, in U.S. Patent Application Publication Nos. 2022/0233382 A1 and 2022/0313515 A1, each of which is hereby incorporated by reference herein in its entirety for all that it teaches to the extent not inconsistent with the present disclosure which shall control as to any inconsistencies.

Microcontroller 50 of MCB 36 serves as the main microcontroller 50 of bed 14 and the microcontrollers of ACB 38, SCB 40, PCB 42, and communications board 44, as well as any microcontrollers of user inputs 46 (e.g., a microcontroller of the GUI of bed 14) and/or any separate microcontroller associated with sensor 48 (e.g., for processing of physiological signals), are considered subordinate microcontrollers of bed 14. Service updates transmitted from service tool 12 that pertain to any of these subordinate microcontrollers, are transmitted first to microcontroller 50 of MCB 36 for storage in memory 54 during a service communication session. After completion of the service communication session, the main microcontroller 50 is configured to promulgate the service updates out to corresponding ones of the subordinate microcontrollers to which the service updates pertain. Thus, the service updates are promulgated out to ACB 38, SCB 40, PCB 42, and communications board 44 for storage in respective memories 64, 68, 74, 82, for example. Similarly, any service updates pertaining to user inputs 46 and/or sensor 48 are promulgated out to the associated microcontrollers of user inputs 46 and/or sensor 48. As alluded to above, the service updates include software code, operating parameters, and the like. Thus, the software updates overwrite and replace the existing software code, operating parameters, etc., as the case may be, in some embodiments.

As mentioned above, the MCB 36, ACB 38, SCB 40, PCB 42, and communications board 44, as well as any microcontrollers of user inputs 46 and/or any separate microcontroller associated with sensor 48 are networked together via a CAN bus in some embodiments. However, it within the scope of the present disclosure for other types of communications links to interconnect MCB 36, ACB 38, SCB 40, PCB 42, and communications board 44, as well as any microcontrollers of user inputs 46 and/or any separate microcontroller associated with sensor 48. Accordingly, in some embodiments, the main microcontroller 50 is configured to promulgate the service updates out to corresponding ones of the subordinate microcontrollers via any one or more of the following protocols: SPI, I²C, CAN, USB, RS-232, and RS-485, just to name a few examples. Some beds 14 contemplated by the present disclosure use some or all of these communication protocols over the respective on-board communications links of the same bed 14. For example, the model no. i.MX35 processor available from Freescale Semiconductor, Inc. of Austin, Texas, U.S.A. may be used as the main microcontroller 50 of bed 14 in some embodiments and the i.MX35 processor supports communications according to a variety of communication protocols including some of those just listed.

The present disclosure also contemplates that, in some embodiments, service tool 12 provides one or more service updates for bed data communications module 90 through bed 14. Thus, during the service communication session in such embodiments, the service update(s) for module 90 are communicated first to MCB 36 over wireless side channel 16 for storage in memory 54. After the service communication session, MCB 36 transmits the one or more service update(s) for module 90 to communications board 44 for storage in memory 82. Microprocessor 80 of communications board 44 then uses BT transceiver 84 to send the one or more service updates for module 90 to BT transceiver 98 over BT channel 88 for storage in memory 96. The bed data communications module 90 then operates according to the received service update(s). In some embodiments, communications board 44 of bed 14 is omitted and BT radio 84 is included in MCB 36. In such embodiments, MCB 36 sends the service updates to module 90 using the integrated BT radio 84 and either antenna 60, or a dedicated BT antenna (e.g., antenna 86 is also integrated into MCB 36).

By sending the service updates for module 90 through bed 14, the service tool 12 does not need to separately link with module 90, either via a direct wired or wireless communication link, to provide the service updates to module 90, but can instead provide such service updates for module 90 during the service communication session with bed 14 along with the service updates for bed 14. It should be appreciated, therefore, that the service updates for module 90 are first communicated over wireless side channel 16 according to the WiFi Direct protocol, then are communicated over the onboard network of bed 14 from MCB 36 to communications board 44 according to whatever wired communication protocol (e.g., CAN, SPI, I²C, etc.) is used to interconnect them, and then are communicated over BT channel 88 to module 90 according to the BT protocol. Thus, the microcontroller having microprocessors 52, 80 are configured to perform any needed protocol conversion for subsequent transmission of the service updates for module 90.

With continued reference to FIG. 1, nurse call system 92 includes a nurse call server 104, a master nurse station computer 106 that is communicatively coupled to nurse call server 104 via a Power over Ethernet (POE) switch 108. In the illustrative example, nurse call server 104 is also coupled to a status board 110 via POE switch 108 and a facility network switch 112. Status board 110 is, in turn, communicatively coupled via switches 108, 112 to master nurse station computer 106 and an input/output (I/O) board 114 that is packaged as a unit with dome light 116. Each unit, wing, or ward of the healthcare facility has dome lights 28, and therefore, I/O boards 60, for each patient room of the unit. However, only one dome light 116 and one I/O board 114 is shown in FIG. 1 for ease of illustration. Nurse call system 92 further includes a graphical audio station (GAS) 118 (sometimes referred to as a graphical room station (GRS)) in each room that is communicatively coupled to the respective I/O board 114. Nurse call system 92 still further includes an audio station bed connector (ASBC) or bed interface unit (BIU) 120 in each room that is communicatively coupled to the respective I/O board 114 and that is communicatively coupled to the patient bed 14 in the respective room. For example, ASBC/NIU 120 has a port (e.g., 37-pin connector) to which cable 102 from bed data communication module 90 couples or to which the nurse call cable from bed 14 couples.

The present disclosure contemplates that, in some embodiments, details pertaining to the service update status of one or more beds 14 are presented to caregivers for viewing on a display of master nurse station computer 106 and/or on status board 110. For example, an icon or other visual indicator is displayed while the respective bed 14 is actively in communication with service tool 12 during a communication session. As another example, if an error is encountered during the service update process, an alarm icon is displayed on station 106 and/or status board to indicate the service update error for the respective bed 14. Information pertaining to software versions installed on each bed also is displayed at station 106 and/or on status board 110 in some embodiments. Nurse call system 92, such as server 104, is also communicatively coupled to facility network 18 via appropriate infrastructure as indicated diagrammatically in FIG. 1 via double headed arrow 122. Thus, service update status, errors and alerts, and the other status update information just mentioned, is communicated from nurse call system 92 to other computer devices of facility network 18 in some embodiments of system 10.

As further shown in FIG. 1, facility network 18 is coupled through a firewall 124 and the Internet 126 to a remote service server 128 which, in turn, is coupled to one or more remote service computers 130. Remote service server 128 and the associated computers 130 are located, for example, at a facility of a manufacturer of bed 14 and/or the components of nurse call system 92. The service technician carrying service tool 12 may be an employee of the manufacturer as well. In any event, the service update status, errors and alerts, and the other status update information available for viewing at master nurse station computer 106, status board 110, and/or computers of facility network 18 as well as on display 28 of service tool 12, is also communicated to remote service server 128 via firewall 124 and Internet 126 for storage and for viewing on a display screen of the one or more remote service computers 130 in some embodiments of system 10.

The present disclosure also contemplates that service update status, alerts and errors, and other service update information is communicated from bed 14 to facility network 18 over primary wireless channel 20 and the associated WAP 22 in addition to, or in lieu of, such information being communicated to facility network 18 by nurse call system 92. A bed identification (ID) or location ID is transmitted along with the service update information so that computer devices of system 10 is able to associate the service update information with the corresponding bed 14 and/or location. This same type of service information is transmitted from service tool 12 to facility network 18 via the associated WAP 22 in addition to, or in lieu of, such information being communicated to facility network 18 by bed 14 and/or nurse call system 92.

With regard to sensor 48 and other sensors of bed 14, such as load cells 70a, 70b, 70c, 70d, the patient parameters sensed thereby, such as patient HR, RR, and weight, falls into the category of protected health information (PHI). The present disclosure contemplates that such PHI is not accessible or available to service tool 12. That is, MCB 36 is configured such that no PHI can be transmitted to service tool 12 over wireless side channel 16. This can be done by storing the PHI in a separate memory cache of memory 54 of SoM 50 or by storing the PHI in a separate memory chip (not shown) of MCB 36. The separate memory cache of memory 54 and/or the separate memory chip are blocked from access by service tool 12. This prevents a service technician, who is most likely not a healthcare provider or even an employee of the healthcare facility in which bed 14 is located, from gaining access to the PHI of any patient supported on bed 14. Of course, such PHI is available to other computer devices of the facility network 18 and nurse call system 92. Thus, the PHI of the patient on bed 14 is transmittable over wireless primary channel 20 and over BT channel 88, but not over wireless side channel 16 according to the preset disclosure.

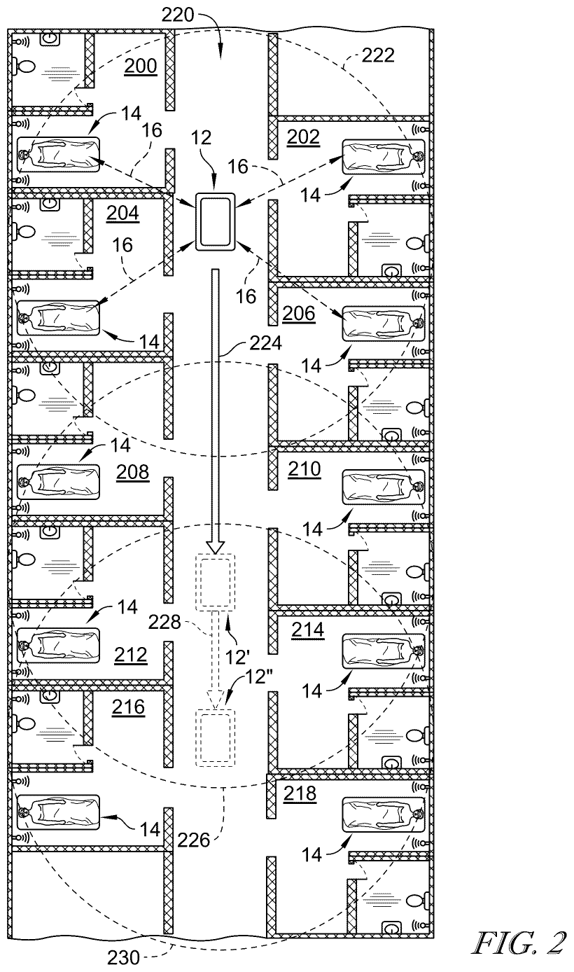

Referring now to FIG. 2, a diagrammatic portion of a floorplan of the healthcare facility is provided showing service tool 12 at a first position (in solid) in a hallway 220 and in wireless communication range of four patient beds 14. The communication range of service tool 12 on the floorplan of FIG. 2 is shown diagrammatically (in phantom) with a circle 222. It should be appreciated that it is possible for service tool 12 to able to communicate wirelessly with patient beds 14 that are on floors above and below the floor depicted in FIG. 2. Thus, the full communication range of service tool 12 is a volume of space (e.g., a sphere or some other non-spherical volume including an irregular volume defined by the geometry of antenna 32 of service tool 12 and influenced by the objects, including walls, floors, ceilings, and equipment through which the wireless signals between service tool 12 and bed 14 must pass). However, within the plan of the floorplan of FIG. 2, the communication range of service tool 12 is shown diagrammatically in FIG. 2 as circle 222 for ease of illustration.

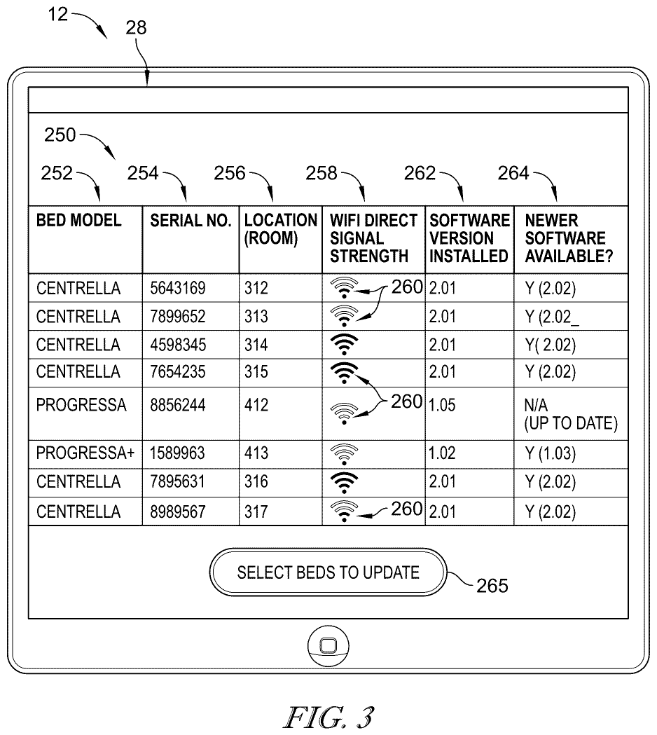

In FIG. 2, there are ten patient rooms shown, namely, rooms 200, 202, 204, 206, 208, 210, 212, 214, 216, 218, each having one of patient beds 14 therein. In some healthcare facilities having semi-private rooms, there are two patient beds 14 per room. Furthermore, an intensive care unit (not shown) may have multiple beds 14 (e.g., five beds 14, ten beds 14, etc.) therein. Services tool 12 is able to provide service updates to a threshold number of beds, such as three beds 14, four beds 14, etc., in such arrangements. In FIG. 2, when service tool 12 is located at the first position in hallway 220, wireless side channels 16 are formed between service tool 12 and four beds 14, namely, the beds 14 in rooms 200, 202, 204, 206. These four beds 14 are the ones within circle 222 which depicts the communication range of the Wireless Direct communication functionality of service tool 12 on the depicted floor of the floorplan. As will be discussed below in connection with FIGS. 3-6, the service technician provides inputs to display 28 of service tool 12 to select which beds 14 from among those within the communication range of tool 12 are to receive service updates.

As indicated diagrammatically by arrow 224 in FIG. 2, after the selected one or more beds 14 have received the respective service updates over corresponding wireless side channels 16 when service tool 12 is at the first position, the service technician transports or carries the service tool 12 to a second position within hallway 220. In FIG. 2, the service tool is designated by reference number 12′ in the second position. In FIG. 2, when service tool 12′ is located at the second position in hallway 220, wireless side channels 16 are formed between service tool 12′ and four beds 14, namely, the beds 14 in rooms 208, 210, 212, 214. These four beds 14 are the ones within a circle 226 which depicts the communication range of the Wireless Direct communication functionality of service tool 12′ in the second position on the depicted floor of the floorplan. The service technician then provides inputs to display 28 of service tool 12′ in the second position to select which beds 14 from among those within the communication range of tool 12′ are to receive service updates.

As indicated diagrammatically by phantom arrow 228 in FIG. 2, after the selected one or more beds 14 have received the respective service updates over corresponding wireless side channels 16 when service tool 12′ is at the second position, the service technician transports or carries the service tool 12′ to a third position within hallway 220. In FIG. 2, the service tool is designated by reference number 12′ in the third position. In FIG. 2, when service tool 12′ is located at the second position in hallway 220, wireless side channels 16 are formed between service tool 12′ and four beds 14, namely, the beds 14 in rooms 212, 214, 216, 218. These four beds 14 are the ones within a circle 230 which depicts the communication range of the Wireless Direct communication functionality of service tool 12′ in the third position on the depicted floor of the floorplan.

When service tool 12′ is in the third position, the service technician then provides inputs to display 28 of service tool 12′ in the third position to select which beds 14 from among those within the communication range of tool 12′ are to receive service updates. Presumably, the beds 14 in rooms 212, 214 would have received any service updates while service tool 12′ was in the second position, and so, once service tool 12′ is in the third position, only the beds 14 in rooms 216, 218 would be expected to potentially need service updates. However, if the beds in rooms 212, 214 did not receive service updates while service tool 12′ was in the second position, the beds in rooms 212, 214 could receive the respective service updates while service tool 12′ is in the third position.

Based on the foregoing discussion of FIG. 2, it should be appreciated that the service technician carries or otherwise transports service tool 12 around the healthcare facility and provides inputs on display 28 of service tool 12 to provide service updates to a threshold number of beds 14 via respective wireless side channels 16. This can be accomplished without the service technician needing to enter any of the patient rooms of the healthcare facility. Of course, this does not exclude the possibility that the service technician may enter a patient room with service tool 12 from time-to-time as desired and provide service updates to one or more beds 14 while located within a particular patient room of the healthcare facility.

As discussed above, the service updates from service tool 12 are provided to patient beds 14 over respective wireless side channels 16 while the patient beds 14 continue to communicate with the facility network 18 over wireless primary channels 20 according to a time slicing protocol. The present disclosure contemplates that circuitry 34 of each bed 14 is able to turn off the time slicing protocol when bed 14 is in communication with service tool 12 over the respective wireless side channel 16. For example, circuitry 34 of the patient support apparatus 14 includes a patient presence detector (e.g., sensor 48 and/or load cells 70a-70d and/or some other patient presence sensor such as a capacitive sensor, optical sensor such as a camera or infrared sensor, force sensitive resistor (FSR), or the like) that produces one or signals that are used by an associated microcontroller/microprocessor to determine whether a patient is present on the patient support apparatus 14 or absent from the patient support apparats 14.

If the patient is absent from the patient support apparatus 14 while the service tool is in communication with the microcontroller via the WiFi Direct communication link of wireless side channel 16, the time slicing protocol is turned off automatically by microcontroller 50, in some embodiments, such that antenna 60 is dedicated for WiFi Direct communications over wireless side channel 16 with service tool 12. In some embodiments, service tool 12 is configured to permit the service technician to turn the WiFi communications of the WiFi communication link 20 on and off based on commands that are entered using display 28, for example, and that are sent from the service tool 12 to microcontroller 50 of bed 14 via the WiFi Direct communication link 16. When the time slicing protocol is turned off such that no communications occur over wireless primary channel 20, the service updates are able to be communicated from service tool 12 to bed 14 more rapidly. It is within the scope of the present disclosure for some beds 14 receiving service updates from service tool 12 while service tool 12 is in a particular position (e.g., first, second, or third positions of FIG. 2), to have the respective time slicing protocols turned off while others of the beds 14 receiving service updates from service tool 12 to have the respective time slicing protocols turned on.

Referring now to FIG. 3, an example of a first screen shot that appears on display 28 of service tool 12 during use is shown. In the first screen shot, a table 250 of information regarding patient beds 14 which are within the communication range over respective side channels 16 of service tool 12 is provided. Table 250 includes a BED MODEL column 252 that indicates the model names of beds 14 with which service tool 12 is communicating over channels 16. In the illustrative example of FIG. 3, the bed models include the CENTRELLA® bed (rows 1-4, 7, and 8 under the BED MODEL heading), the PROGRESSA®′bed (row 5), and the PROGRESSA+® bed (row 6), each of which is available from Hill-Rom Company, Inc. of Batesville, Indiana, U.S.A. To the right of column 252 in table 250 is a SERIAL NO. column 254 that lists the serial numbers of each of the respective beds listed in table 250.

To the right of column 254 in table 250 is a LOCATION (ROOM) column 256 which indicates the room in which each of the respective beds listed in table 250 are located. Presumably, the beds corresponding to rows 1-4, 7, and 8 are on the third floor of the corresponding healthcare facility, whereas those corresponding to rows 5 and 6 are on the fourth floor of the corresponding healthcare. To the right of column 256 in table 250 is a WIFI DIRECT SIGNAL STRENGTH column 258 which indicates a signal strength of the communications over the respective wireless side channels 16 for each of the listed patient beds. In the illustrative example, the signal strengths of wireless side channels 16 are indicated in table 250 with signal strength icons 260 which each include a dot and three curved bars above the dot. The number of curved bars that are filled in indicate whether the signal strength is poor (e.g., no curved bars filled in), fair (e.g., one curved bar filled in), good (e.g., two curved bars filled in), or excellent (e.g., all three curved bars filled in). In the illustrative FIG. 3 example, the signal strengths of the channels 16 corresponding to rows 5 and 6 is poor, the signal strengths of the channels 16 corresponding to rows 1, 2 and 8 are fair, and the signal strengths of the channels 16 corresponding to rows 3, 4, and 7 are excellent.

Continuing from left to right in table 250 of FIG. 3, a SOFTWARE VERSION INSTALLED column 262 is provided to the right of column 258. Each row of column 262 indicates the software version number installed in the corresponding bed 14. To the right of column 262 in table 250 is a NEWER SOFTWARE AVAILABLE? column 264. Each row of columns 264 includes a “Y” if there is a new software version available and the newer software version in parentheses adjacent to the “Y.” If no newer version of software is available, then “N/A (UP TO DATE)” appears in the corresponding row of column 264. In the illustrative example, a new version of software is available for the beds 14 associated with rows 1-4 and 6-8 and a new version of software is not available for the bed associated with row 5. The updated software versions correspond to service updates needed for any one or more of MCB 36, ACB 38, SCB 40, PCB 42, communications board 44, user inputs 46, and HR/RR sensor 48, for example.

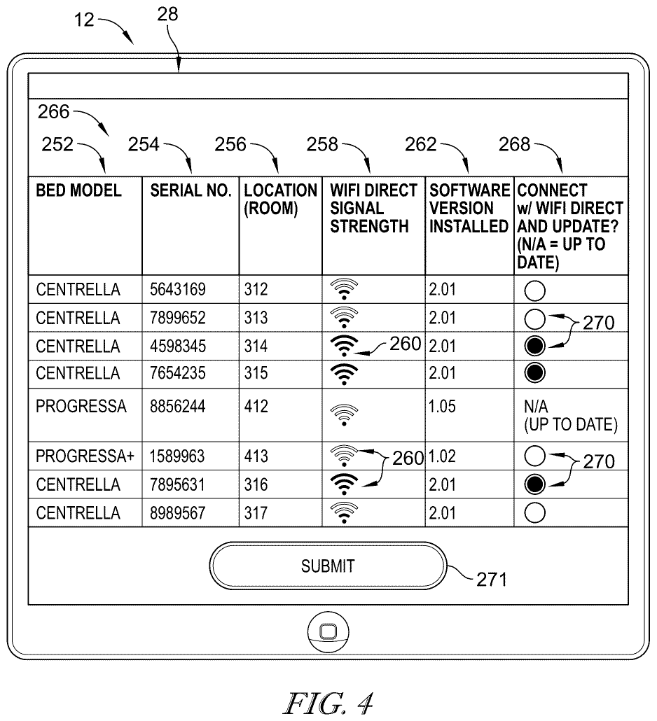

Beneath table 250 in FIG. 3 is a SELECT BEDS TO UPDATE button or input 265 which is selected by the service technician if any of the beds listed in column 252 are to be updated with a service update. Referring now to FIG. 4, an example is shown of a second shot that appears on display 28 of service tool 12 in response to selection of button 265 of the first screen shot. In response to selection of button 265, the service tool 12 responds by displaying a table 266 on display 28. Table 266 of FIG. 4 includes the same columns 252, 254, 256, 258, 262 that appeared on table 250 of FIG. 3 and so, the descriptions of these columns 252, 254, 256, 258, 262 are equally applicable to table 266 and are not repeated.

To the right of column 262 of table 266 of FIG. 4 is a CONNECT w/WIFI DIRECT AND UPDATE? (N/A=UP TO DATE) column 268. Column 268 includes, in reach row, a radio button 270 that is selectable by the service technician to indicate which of the corresponding beds 14 is to receive a service update from service tool 12 over the respective wireless side channel 16. Thus, initially, each of radio buttons 270 in table 266 is blank and then becomes filled in upon selection by the service technician. Accordingly, in FIG. 4, the radio buttons 270 of rows 3, 4, and 7 of column 268 of table 266 are filled in to indicate that the service technician has selected the beds corresponding to rows 3, 4, and 7 for service updates.

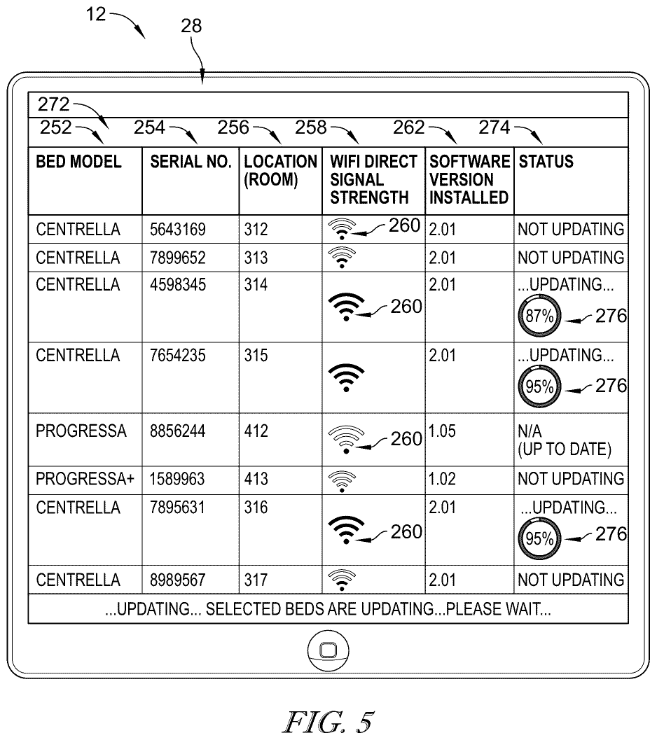

Beneath table 266 in FIG. 4 is a SUBMIT input or button 271 that is selected by the service technician after the desired radio button(s) 270 in column 268 have been selected. In response to selection of button 271, service tool 12 transmits the service updates (e.g., new software versions) to the corresponding beds 14 that have been selected. Referring now to FIG. 5, an example is shown of a third shot that appears on display 28 of service tool 12 in response to selection of button 271 of the second screen shot. In response to selection of button 271, the service tool 12 responds by displaying a table 272 on display 28. Table 272 of FIG. 5 includes the same columns 252, 254, 256, 258, 262 that appeared on tables 250, 266 of FIGS. 3 and 4, respectively, and so, the descriptions of these columns 252, 254, 256, 258, 262 are equally applicable to table 272 and are not repeated.

To the right of column 262 of table 272 of FIG. 5 is a STATUS column 274 which includes, in reach row, either the text “NOT UPDATING” for any beds that are not receiving services updates or the text “ . . . UPDATING” for any beds that are receiving service updates. Furthermore, in each row of any beds 14 that are receiving service updates, a progress indicator 276 is provided to show the progress of the transmission (aka upload) of the corresponding service update to the respective bed 14. In the illustrative example, each progress indicator 276 appears beneath the text “ . . . UPDATING” and includes a circle that fills in by an amount corresponding to the upload progress and also includes, within the circle, a numerical per centage value corresponding to the upload progress. Thus, each progress indicator 276 is a dynamic indicator or icon that changes the amount that the circle fills in and that changes the numerical per centage value as the service update is transmitted to the corresponding patient bed 14. In table 272, progress indicators 276 appear in rows 3, 4, and 7 because those are the rows for which radio buttons 270 were selected on table 266 of FIG. 4 prior to selection of button 271. In the illustrative example of FIG. 5, the text “ . . . Updating . . . Selected Beds Are Updating . . . Please Wait . . . ” appears beneath table 272 in FIG. 5.

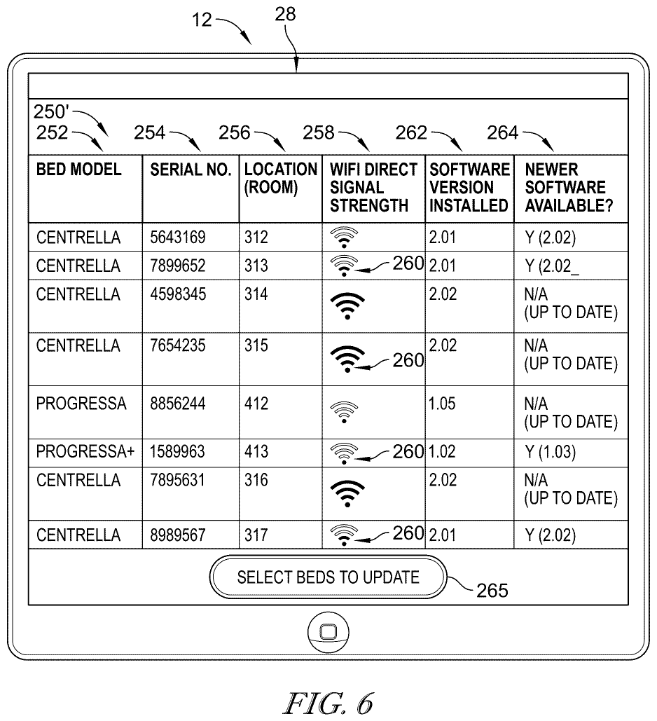

Referring now to FIG. 6, an example of a fourth screen shot that appears on display 28 of service tool 12 after the service update process has been completed for the selected patient beds 14 is shown. The fourth screen shot of FIG. 6 includes a table 250′ which is substantially similar to table 250 of FIG. 3 except that column 264 indicates that the beds corresponding to rows 3, 4, and 7 are now up to date as concerns the software version installed. Thus, table 250′ of FIG. 6 includes the same columns 252, 254, 256, 258, 262, 264 as table 250 of FIG. 3 but just with updated information in column 264. Of course, the service technician can select button 265 beneath table 250′ and then select other beds 14 to have service updates installed. In the FIG. 6 example, column 264 of table 250′ indicates that the beds 14 associated with rows 1, 2, 6, and 8 have newer versions of software available for installation from service tool 12.

In some contemplated embodiments, one of the user inputs 46 of patient support apparatus 14 is selectable to signal the circuitry 34 of patient support apparatus 14 to enter into a service tool discovery mode to link with the service tool 12 via the wireless communication link provided by wireless side channel 16. Such a user input includes a dedicated service tool discovery button provided on a control panel of bed 14 (e.g., a hard button) or rendered on the GUI of bed 14 (e.g., a soft button). In other contemplated embodiments, patient support apparatus 14 includes a plurality of user inputs, such as hard buttons and/or soft buttons like those just described that, in response to being selected in a predetermined sequence, causes the circuitry 34 to enter into the service tool discovery mode to link with the service tool 12 via the wireless communication link provided by the wireless side channel 16. In still further contemplated embodiments, patient support apparatus 14 includes a plurality of user inputs, such as hard buttons and/or soft buttons like those previously described that, in response to being selected simultaneously, causes circuitry 34 to enter into a service tool discovery mode to link with the service tool 12 via the wireless communication link provided by wireless side channel 16.

When terms of degree such as “generally,” “substantially,” and “about” are used herein in connection with a numerical value or a qualitative term susceptible to a numerical measurement, it is contemplated that an amount that is plus or minus 10 percent, and possibly up to plus or minus 20 percent, of the numerical value, is covered by such language, unless specifically noted otherwise, to at least account for manufacturing tolerances. Otherwise, a suitable definition for “generally,” “substantially,” and “about” is largely, but not necessarily wholly, the term specified.

When the terms “a” or “an” or the phrases “one or more” or “at least one” are used herein, including in the claims, they are all intended to be synonymous and mean that one, or more than one, of the thing recited may be present. Similarly, when the phrases “a plurality” or “two or more” or “at least two” or “a pair” are used, they are all intended to be synonymous and mean that two, or more than two, of the thing recited may be present.

According to this disclosure, phrases of the form “at least one of A and B” and “at least one of the following: A and B” and similar such phrases, mean “A alone, or B alone, or both A and B.” Phrases of the form “at least one of A or B” and “at least one of the following: A or B” and similar such phrases, also mean “A alone, or B alone, or both A and B.” Furthermore, phrases of the form “A and/or B” also mean “A alone, or B alone, or both A and B.”

Although certain illustrative embodiments have been described in detail above, variations and modifications exist within the scope and spirit of this disclosure as described and as defined in the following claims.

1. A system for use in a healthcare facility having a network including a plurality of wireless access points, the system comprising
a patient support apparatus having circuitry including a microcontroller configured to communicate wirelessly via a first communication link with at least one wireless access point of the plurality of wireless access points, the microcontroller being configured to execute operating instructions to control one or more functions of the patient support apparatus, the circuitry of the patient support apparatus including at least one sensor communicatively coupled to the microcontroller and configured to detect at least one patient parameter that is classified as protected health information (PHI), and
a service tool that is configured to communicate wirelessly with the microcontroller via a second communication link to provide service updates to the circuitry of the patient support apparatus, wherein the circuitry is configured to maintain the first communication link during communications with the service tool via the second communication link thereby permitting a patient to remain supported on the patient support apparatus while the service updates are provided to the circuitry of the patient support apparatus, wherein the microcontroller is configured so that the PHI is wirelessly transmittable from the patient support apparatus via the first communication link but not via the second communication link.

2. The system of claim 1, wherein the first communication link comprises a WiFi communication link and the second communication link comprises a WiFi Direct communication link.

3. The system of claim 2, wherein the microcontroller is included in a system on a module (SoM) having an antenna that is used for both WiFi and WiFi Direct communications according to a time slicing protocol.

4. The system of claim 3, wherein the circuitry of the patient support apparatus includes a patient presence detector to determine whether the patient is present on the patient support apparatus or absent from the patient support apparats, and wherein if the patient is absent from the patient support apparatus while the service tool is in communication with the microcontroller via the WiFi Direct communication link, the time slicing protocol is turned off automatically by the microcontroller such that the antenna is dedicated for WiFi Direct communications with the service tool.

5. The system of claim 2, wherein the service tool is configured to permit a service technician to turn the WiFi communications of the WiFi communication link on and off based on commands sent from the service tool via the WiFi Direct communication link.

6. The system of claim 1, wherein the microcontroller serves as a main microcontroller and wherein the circuitry of the patient support apparatus comprises a plurality of subordinate microcontrollers, wherein the service updates are communicated to the main microcontroller from the service tool during a service communication session and then, after completion of the service communication session, the main microcontroller is configured to promulgate the service updates out to corresponding ones of the subordinate microcontrollers to which the service updates pertain.

7. The system of claim 1, further comprising a bed data communication module spaced from the patient support apparatus and mounted at a fixed location in a patient room in which the patient support apparatus is located, the bed data communication module being in wireless communication with the circuitry of the patient support apparatus via a third communication link and being in communication with a nurse call system of the healthcare facility, wherein the service tool is configured to communicate service updates for the bed data communication module to the microcontroller of the patient support apparatus during a service communication session and then, after completion of the service communication session, any service updates for the bed data communication module are sent to the bed data communication module via the third communication link.

8. The system of claim 1, wherein the patient support apparatus includes a user input that is selected to signal the circuitry to enter into a service tool discovery mode to link with the service tool via the second communication link.

9. The system of claim 1, wherein the patient support apparatus includes a plurality of user inputs that, in response to being selected in a predetermined sequence or being selected simultaneously, causes the circuitry to enter into a service tool discovery mode to link with the service tool via the second communication link.

10. A system for use in a healthcare facility having a network including a plurality of wireless access points, the system comprising
a patient support apparatus having circuitry including a microcontroller configured to communicate wirelessly via a first communication link with at least one wireless access point of the plurality of wireless access points, the microcontroller being configured to execute operating instructions to control one or more functions of the patient support apparatus, and
a service tool that is configured to communicate wirelessly with the microcontroller via a second communication link to provide service updates to the circuitry of the patient support apparatus, wherein the circuitry is configured to maintain the first communication link during communications with the service tool via the second communication link thereby permitting a patient to remain supported on the patient support apparatus while the service updates are provided to the circuitry of the patient support apparatus, wherein the service tool is configured to permit a service technician to turn wireless communications of the first communication link on and off based on commands sent from the service tool via the second communication link.

11. The system of claim 10, wherein the first communication link comprises a WiFi communication link and the second communication link comprises a WiFi Direct communication link.

12. The system of claim 11, wherein the microcontroller is included in a system on a module (SoM) having an antenna that is used for both WiFi and WiFi Direct communications according to a time slicing protocol.

13. The system of claim 12, wherein the circuitry of the patient support apparatus includes a patient presence detector to determine whether the patient is present on the patient support apparatus or absent from the patient support apparats, and wherein if the patient is absent from the patient support apparatus while the service tool is in communication with the microcontroller via the WiFi Direct communication link, the time slicing protocol is turned off automatically by the microcontroller such that the antenna is dedicated for WiFi Direct communications with the service tool.

14. The system of claim 10, wherein the microcontroller serves as a main microcontroller and wherein the circuitry of the patient support apparatus comprises a plurality of subordinate microcontrollers, wherein the service updates are communicated to the main microcontroller from the service tool during a service communication session and then, after completion of the service communication session, the main microcontroller is configured to promulgate the service updates out to corresponding ones of the subordinate microcontrollers to which the service updates pertain.

15. The system of claim 10, further comprising a bed data communication module spaced from the patient support apparatus and mounted at a fixed location in a patient room in which the patient support apparatus is located, the bed data communication module being in wireless communication with the circuitry of the patient support apparatus via a third communication link and being in communication with a nurse call system of the healthcare facility, wherein the service tool is configured to communicate service updates for the bed data communication module to the microcontroller of the patient support apparatus during a service communication session and then, after completion of the communication session, any service updates for the bed data communication module are sent to the bed data communication module via the third communication link.

16. The system of claim 10, wherein the patient support apparatus includes a plurality of user inputs that, in response to being selected in a predetermined sequence or being selected simultaneously, causes the circuitry to enter into a service tool discovery mode to link with the service tool via the second communication link.

17. A system for use in a healthcare facility having a network including a plurality of wireless access points, the system comprising
a patient support apparatus having circuitry including a microcontroller configured to communicate wirelessly via a first communication link with at least one wireless access point of the plurality of wireless access points, the microcontroller being configured to execute operating instructions to control one or more functions of the patient support apparatus, and
a service tool that is configured to communicate wirelessly with the microcontroller via a second communication link to provide service updates to the circuitry of the patient support apparatus, wherein the circuitry is configured to maintain the first communication link during communications with the service tool via the second communication link thereby permitting a patient to remain supported on the patient support apparatus while the service updates are provided to the circuitry of the patient support apparatus, wherein the circuitry of the patient support apparatus includes an antenna that is used for wireless communications over the first and second communication links according to a time slicing protocol, wherein the circuitry of the patient support apparatus includes a patient presence detector to determine whether the patient is present on the patient support apparatus or absent from the patient support apparats, and wherein if the patient is absent from the patient support apparatus while the service tool is in communication with the microcontroller via the second communication link, the time slicing protocol is turned off such that the antenna is dedicated for wireless communications only with the service tool over the second communication link.

18. The system of claim 17, further comprising a bed data communication module spaced from the patient support apparatus and mounted at a fixed location in a patient room in which the patient support apparatus is located, the bed data communication module being in wireless communication with the circuitry of the patient support apparatus via a third communication link and being in communication with a nurse call system of the healthcare facility, wherein the service tool is configured to communicate service updates for the bed data communication module to the microcontroller of the patient support apparatus during a service communication session and then, after completion of the service communication session, any service updates for the bed data communication module are sent to the bed data communication module via the third communication link.

19. The system of claim 17, wherein the patient support apparatus includes a plurality of user inputs that, in response to being selected in a predetermined sequence or being selected simultaneously, causes the circuitry to enter into a service tool discovery mode to link with the service tool via the second communication link.

20. The system of claim 17, wherein the patient presence detector comprises one or more load cells of a weigh scale system of the patient support apparatus.
